(19) United States
(12) Patent Application Publication
(10) Pub. No.: US 2024/0325966 A1
(43) Pub. Date: Oct. 3, 2024

(54) REACTION SYSTEM

(71) Applicant: IHI Corporation, Tokyo (JP)

(72) Inventors: Kentaro NARIAI, Tokyo (JP); Jun TSUJIKAWA, Tokyo (JP); Kousuke INABA, Tokyo (JP)

(73) Assignee: IHI Corporation, Tokyo (JP)

(21) Appl. No.: 18/738,206

(22) Filed: Jun. 10, 2024

Related U.S. Application Data
(63) Continuation of application No. PCT/JP2023/007030, filed on Feb. 27, 2023.

(30) Foreign Application Priority Data
Apr. 8, 2022 (JP) 2022-064383

(51) Int. Cl. B01D 53/14

(52) U.S. Cl. CPC B01D 53/1475; B01D 2256/24; B01D 2257/504

(57) ABSTRACT

A reaction system includes a carbon dioxide collection unit that collects carbon dioxide by an absorption process or an adsorption process, a reaction unit that generates hydrocarbons from a raw material containing hydrogen and carbon dioxide, and a distillation unit that distills hydrocarbons, wherein hydrocarbons are distilled in the distillation unit by reaction heat generated by generating hydrocarbons in the reaction unit, and carbon dioxide absorbed or adsorbed in the carbon dioxide collection unit is separated by a low-temperature heat medium that has a temperature lower than a high-temperature heat medium being a heat medium introduced to the distillation unit, as a result of consuming part of the reaction heat in the distillation unit.

### CROSS REFERENCE TO RELATED APPLICATIONS

The present application is a continuation application of International Application No. PCT/JP2023/007030, filed on Feb. 27, 2023, which claims priority to Japanese Patent Application No. 2022-064383, filed on Apr. 8, 2022, the entire contents of which are incorporated by reference herein.

### BACKGROUND

### 1. Technical Field

The present disclosure relates to a reaction system.

### 2. Description of the Related Art

Carbon dioxide is considered as a cause of global warming, and there is a growing movement worldwide to reduce emissions of carbon dioxide. As a method of reducing an emission amount of carbon dioxide and utilizing carbon dioxide effectively, there have been known a methanation technique of producing methane from carbon dioxide in an exhaust gas, a Fischer-Tropsch (FT) synthesis technique of synthesizing various hydrocarbons, and the like. A methanation reaction and an FT synthesis reaction are exothermic. Thus, there is a growing trend to effectively utilize heat generated from a methanation reaction.

JP2019-052224A discloses a heat-utilizing gas purification system including a methane synthesis device using hydrogen and carbon dioxide as raw gases and a temperature swing adsorption gas purification device provided to a raw gas introducing path and a generated gas sending path. In the system, a high-temperature regenerated gas introduced to the temperature swing adsorption gas purification device receives reaction heat from the methane gas synthesis device.

### SUMMARY

According to the related art, as the high-temperature regenerated gas introduced to the temperature swing adsorption gas purification device receives the reaction heat from the methane gas synthesis device, energy efficiency can be improved. However, further improvement in energy efficiency is anticipated.

In view of this, an object of the present disclosure is to provide a reaction system with high energy efficiency.

A reaction system according to the present disclosure includes a carbon dioxide collection unit configured to collect carbon dioxide by an absorption process or an adsorption process, a reaction unit configured to generate hydrocarbons from a raw material containing hydrogen and carbon dioxide, and a distillation unit configured to distill hydrocarbons. In the reaction system, hydrocarbons are distilled in the distillation unit by reaction heat generated by generating hydrocarbons in the reaction unit, and carbon dioxide absorbed or adsorbed in the carbon dioxide collection unit is separated by a low-temperature heat medium that has a temperature lower than a high-temperature heat medium being a heat medium introduced to the distillation unit, as a result of consuming part of the reaction heat in the distillation unit.

The distillation unit may distill hydrocarbons being generated in the reaction unit.

Carbon dioxide contained in the raw material may include carbon dioxide being collected in the carbon dioxide collection unit.

The carbon dioxide collection unit may include an absorption unit configured to absorb carbon dioxide into an absorption solution and a separation unit configured to separate, from the absorption solution, carbon dioxide being absorbed into the absorption solution, and the separation unit may separate carbon dioxide by the low-temperature heat medium.

The reaction unit may include a fixed bed reactor.

The distillation unit may include at least one of an atmospheric distillation unit and a vacuum distillation unit, and may distill hydrocarbons by the reaction heat, the hydrocarbons being introduced to at least one of the atmospheric distillation unit and the vacuum distillation unit.

According to the present disclosure, it is possible to provide a reaction system with high energy efficiency.

### BRIEF DESCRIPTION OF THE DRAWINGS

FIG. 1 is a schematic diagram illustrating a reaction system according to one embodiment.

FIG. 2 is a schematic diagram illustrating a heat medium flow passage of the reaction system according to one embodiment.

FIG. 3 is a schematic diagram illustrating the heat medium flow passage of the reaction system according to one embodiment.

### DESCRIPTION OF THE EMBODIMENTS

Some exemplary embodiments are described below with reference to the drawings. Note that the dimension ratios in the drawings are exaggerated for the convenience of the description, and may be different from the actual ratios.

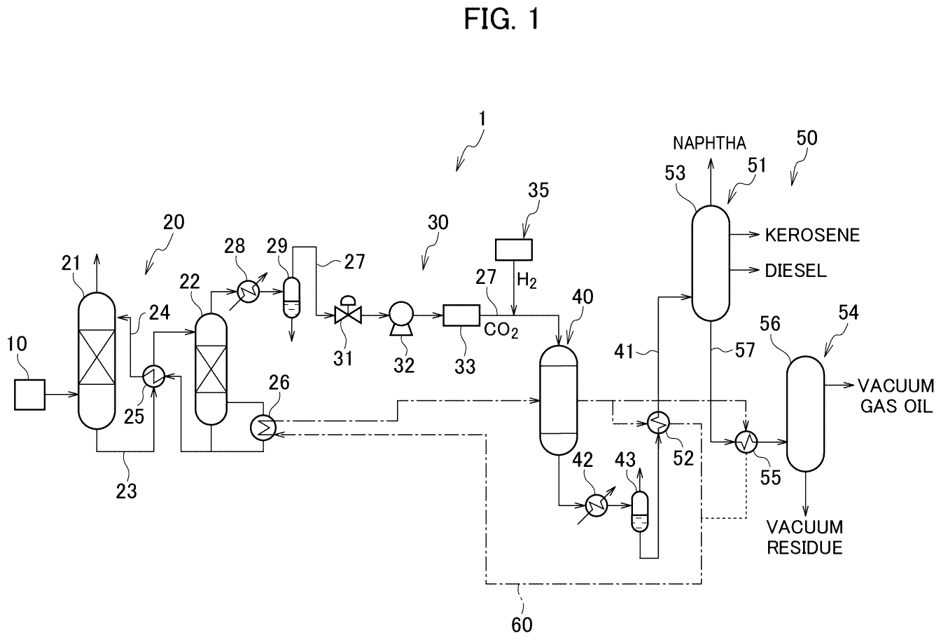

As illustrated in FIG. 1, a reaction system 1 according to the present embodiment includes a carbon dioxide generation source 10, a carbon dioxide collection unit 20, a carbon dioxide supply unit 30, a hydrogen supply unit 35, a reaction unit 40, and a distillation unit 50.

For example, the carbon dioxide generation source 10 is a power plant, a factory, or the like that emits carbon dioxide through combustion of fuel. The carbon dioxide generation source 10 may include a boiler.

The carbon dioxide collection unit 20 collects carbon dioxide generated from the carbon dioxide generation source 10. The carbon dioxide collection unit 20 is capable of reducing an amount of carbon dioxide released into the atmosphere by collecting carbon dioxide generated from the carbon dioxide generation source 10.

The carbon dioxide collection unit 20 may collect carbon dioxide by a chemical absorption process. As illustrated in FIG. 1, the carbon dioxide collection unit 20 may include an absorption unit 21, a separation unit 22, a supply pipe 23, a reflux pipe 24, a heat exchanger 25, a reboiler 26, a cooling unit 28, and a gas-liquid separation unit 29. The supply pipe 23 connects a lower portion of the absorption unit 21 and an upper portion of the separation unit 22 to each other. The reflux pipe 24 connects a lower portion of the separation unit 22 and an upper portion of the absorption unit 21 to each other. The supply pipe 23 and the reflux pipe 24 are provided with the heat exchanger 25.

The absorption unit 21 absorbs carbon dioxide into an absorption solution. Specifically, the absorption unit 21 absorbs carbon dioxide through gas-liquid contact between a gas containing carbon dioxide and the absorption solution. In the present embodiment, the absorption unit 21 is an absorption column. The separation unit 22 separates, from the absorption solution, carbon dioxide absorbed into the absorption solution. In the present embodiment, the separation unit 22 is a releasing column. The absorption solution may be an alkaline solution. Specifically, the absorption solution may contain at least one of alkanolamine and hindered amine containing an alcoholic hydroxy group. More specifically, the absorption solution may contain monoethanolamine (MEA).

A gas containing carbon dioxide that is supplied from a lower part of the absorption unit 21 is brought into gas-liquid contact with the absorption solution, and carbon dioxide contained in the gas is absorbed into the absorption solution. The absorption solution absorbing carbon dioxide passes through the supply pipe 23, is heated by the heat exchanger 25, and then is transferred upward to the separation unit 22. The absorption solution that is heated by the heat exchanger 25 drips down from an upper part of the separation unit 22 while releasing carbon dioxide, and stagnates at a bottom portion of the separation unit 22. The absorption solution stagnating at the bottom portion of the separation unit 22 is heated by the reboiler 26, and carbon dioxide is released from the absorption solution. A gas containing released carbon dioxide is discharged through a gas discharge port provided in the top of the separation unit 22.

Meanwhile, the absorption solution stagnating at the bottom portion of the separation unit 22 passes through the reflux pipe 24, is cooled by the heat exchanger 25, and then is transferred to the upper portion of the absorption unit 21. In this state, the absorption solution passing through the supply pipe 23 and the absorption solution passing through the reflux pipe 24 exchange heat to heat the absorption solution passing through the supply pipe 23 and cool the absorption solution passing through the reflux pipe 24. The absorption solution that is supplied from above a filling material in the absorption unit 21 is brought into gas-liquid contact with the gas containing carbon dioxide that is supplied from the carbon dioxide generation source 10, and carbon dioxide is absorbed into the absorption solution again. The gas from which carbon dioxide is removed in the absorption unit 21 is discharged through a gas discharge port provided in the top of the absorption unit 21.

The gas discharge port of the separation unit 22 and the reaction unit 40 are connected to each other via a pipe 27. The pipe 27 is provided with a cooling unit 28, a gas-liquid separation unit 29, and the carbon dioxide supply unit 30. The gas containing carbon dioxide that is discharged through the gas discharge port of the separation unit 22 passes through the pipe 27, and is cooled by the cooling unit 28. Then, moisture contained in the gas and the absorption solution are condensed. The condensed water and the like are separated in the gas-liquid separation unit 29, and is returned to the separation unit 22 via a pipe, which is omitted in the illustration. The gas separated from the carbon dioxide collection unit 20 contains, for example, 90% or more, 95% or more, or 99% or more carbon dioxide by mass. The carbon dioxide supply unit 30 includes a flow rate adjustment unit 31, a compressor 32, and a flow rate adjustment unit 33.

The flow rate adjustment unit 31 is provided downstream of the cooling unit 28 and the gas-liquid separation unit 29 in the pipe 27. The flow rate adjustment unit 31 adjusts a flow rate of a gas flowing through the pipe 27.

The compressor 32 compresses a gas. The gas compressed by the compressor 32 includes carbon dioxide separated from the carbon dioxide collection unit 20. The compressor 32 has an inlet and an outlet, and the gas sucked in through the inlet is compressed and discharged through the outlet.

The flow rate adjustment unit 33 adjusts a flow rate of the gas compressed by the compressor 32. The flow rate adjustment unit 33 adjusts a flow rate of the gas that is compressed by the compressor 32 and is supplied to the reaction unit 40. The flow rate adjustment unit 33 may include a mass flow controller.

The hydrogen supply unit 35 supplies hydrogen to the reaction unit 40. The hydrogen supply unit 35 is not particularly limited as long as hydrogen can be supplied to the reaction unit 40, and may utilize renewable energy sources such as solar power, wind power, and hydro power and use hydrogen obtained from the electrolysis of water. When hydrogen thus obtained is used, an emission amount of carbon dioxide from the entire reaction system 1 can be reduced.

A raw material containing hydrogen and carbon dioxide is supplied to the reaction unit 40. In the present embodiment, carbon dioxide contained in the raw material includes carbon dioxide that is collected in the carbon dioxide collection unit 20. With this, it is possible to provide the reaction system 1 in which the carbon dioxide collection unit 20 and the reaction unit 40 are integrated with each other. However, the reaction unit 40 may be independent from the carbon dioxide collection unit 20, and may use, in the raw material, carbon dioxide that does not pass through the carbon dioxide collection unit 20. The raw material supplied to the reaction unit 40 may be heated by a heating unit, which is omitted in the illustration. The amount ratio of hydrogen to carbon dioxide supplied to the reaction unit 40 can be appropriately adjusted. For example, in terms of molar ratio, the amount ratio may be 1 or more, 2 or more, 3 or more, 3.5 or more, or 4 or more. Further, for example, in terms of molar ratio, the amount ratio of hydrogen to carbon dioxide supplied to the reaction unit 40 may be less than 8, less than 6, less than 5, or less than 4.5. Note that, in a case of a methanation reaction, the amount ratio of hydrogen to carbon dioxide supplied to the reaction unit 40 may be 4 being a stoichiometry ratio.

The reaction unit 40 generates hydrocarbons from the raw material containing hydrogen and carbon dioxide. By generating hydrocarbons in the reaction unit 40, not only can carbon dioxide emissions be reduced, but carbon dioxide can also be effectively utilized.

Hydrocarbons may contain at least one of an alkane and an alkene. These hydrocarbons can be generated by a methanation reaction or a Fischer-Tropsch (FT) reaction. At least one of an alkane and an alkene may contain hydrocarbons with a carbon number ranging from 1 to 100. For example, at least one of an alkane and an alkene may contain hydrocarbons with a carbon number ranging from 1 to 4. Examples of an alkane with a carbon number ranging from 1 to 4 include methane, ethane, propane, and butane. Examples of an alkene with a carbon number ranging from 1 to 4 include ethylene, propylene, 1-butene, 2-butene, isobutene, and 1,3-butadiene. Note that, among those, methane, ethane, and propane can be used as fuels for city gas. Further, an alkene with a carbon number ranging from 2 to 4 is useful because it can also serve as a raw material for plastics. Note that a reaction product may contain compounds other than those mentioned above.

The reaction unit 40 may include a fixed bed reactor. The fixed bed reactor has a relatively simple structure, and hence the reaction system 1 with a simple configuration can be provided. The fixed bed reactor may be a single-tube reactor or a multi-tube reactor such as a shell-and-tube type reactor. The fixed bed reactor may include a reaction tube and a shell that accommodates the reaction tube. When a heat medium being a cooling medium passes through the outside of the reaction tube in the shell, reaction heat that is generated by generating hydrocarbons in the reaction unit 40 can be extracted. A catalyst may be arranged inside the reaction tube. The raw material passes through the reaction tube, and is brought into contact with the catalyst. With this, carbon dioxide and hydrogen that are contained in the raw material react to generate hydrocarbons.

A publicly known catalyst such as an iron catalyst and a cobalt catalyst may be used as long as the catalyst is selected based on a type of hydrocarbons to be generated. In a case of the iron catalyst, light hydrocarbons can be mainly generated. In a case of the cobalt catalyst, heavy hydrocarbons containing wax can be mainly generated. Further, in a case of the iron catalyst, an alkene and an alkane can be mainly generated. In a case of the cobalt catalyst, an alkane can be mainly generated. Note that the iron catalyst is a catalyst containing iron as an active component, and the cobalt catalyst is a catalyst containing cobalt as an active component. The reaction conditions in the reaction unit 40 are not particularly limited. However, for example, the reaction temperature ranges from 200 degrees Celsius to 500 degrees Celsius, and the pressure ranges from 0.3 MPaG to 3 MPaG.

The reaction unit 40 and an atmospheric distillation column 53 of the distillation unit 50 are connected to each other via a pipe 41. The pipe 41 is provided with a cooling unit 42, a gas-liquid separation unit 43, and a heating unit 52. The cooling unit 42 cools the reaction product that is generated in the reaction unit 40. With this, part of the reaction product that is generated in the reaction unit 40 can be condensed. The gas-liquid separation unit 43 separates a remaining gaseous low-boiling point reaction product, which is not condensed by the cooling unit 42, from a high-boiling point reaction product, which is condensed by the cooling unit 42 and has a higher boiling point than the low-boiling point reaction product. For example, the low-boiling point reaction product may contain methane. The low-boiling point reaction product may contain ethane, propane, and butane. Further, the high-boiling point reaction product contains a plurality of types of reaction products having different boiling points. The high-boiling point reaction product may contain two or more types of hydrocarbons ranging from C5 to C100. For example, the high-boiling point reaction product may contain hydrocarbons ranging from C5 to C15. Liquid hydrocarbons ranging from C5 to C15 can be used as aviation fuel. The high-boiling point reaction product may also be supplied to the distillation unit 50.

The distillation unit 50 distills hydrocarbons that are generated in the reaction unit 40. With this, the reaction system 1 in which the reaction unit 40 and the distillation unit 50 are integrated with each other can be provided. However, the distillation unit 50 may be independent from the reaction unit 40, and may distill hydrocarbons that are not generated in the reaction unit 40. The distillation unit 50 separates a mixture of hydrocarbons having different boiling points by using a difference between the boiling points. The reaction product that is generated in the reaction unit 40 may be subjected to hydrogenation treatments such as hydrogenation purification, hydrogen isomerization, and hydrogenation decomposition. The distillation unit 50 includes an atmospheric distillation unit 51 and a vacuum distillation unit 54.

The atmospheric distillation unit 51 includes a heating unit 52 and an atmospheric distillation column 53. The heating unit 52 is provided to the pipe 41, and heats the mixture of hydrocarbons that is introduced to the atmospheric distillation column 53. For example, the temperature of the mixture after heating may range from 300 degrees Celsius to 400 degrees Celsius. The atmospheric distillation column 53 distills the mixture of hydrocarbons that is heated by the heating unit 52. The atmospheric distillation column 53 includes a plurality of trays, and is capable of separating hydrocarbons according to the carbon number by extracting a liquid component from each tray.

The atmospheric distillation unit 51 distills the mixture of hydrocarbons having different boiling points under an atmospheric pressure of, for example, approximately 0.5 atm to 2 atm. In the atmospheric distillation unit 51, distillation allows for the separation of various fractions such as naphtha, kerosene, diesel, and residual oil. Naphtha may contain hydrocarbons ranging from C4 to C12, for example. The boiling point of hydrocarbons ranging from C4 to C12 ranges from 35 degrees Celsius to 180 degrees Celsius, for example. Kerosene may contain hydrocarbons ranging from C12 to C18, for example. The boiling point of hydrocarbons ranging from C12 to C18 ranges from 170 degrees Celsius to 250degrees Celsius, for example. Diesel may contain hydrocarbons ranging from C14 to C23, for example. The boiling point of hydrocarbons ranging from C14 to C23 ranges from 240 degrees Celsius to 350 degrees Celsius, for example. The residual oil is used to refer to hydrocarbons that remain after distillation, and may contain hydrocarbons of C17 or higher, for example. The boiling point of hydrocarbons of C17 or higher is 350 degrees Celsius or higher, for example.

The vacuum distillation unit 54 includes a heating unit 55, a vacuum distillation column 56, and a vacuum unit, which is omitted in the illustration. The atmospheric distillation column 53 and the vacuum distillation column 56 are connected to each other via a pipe 57. The residual oil (the atmospheric residual oil) distilled in the atmospheric distillation column 53 is introduced to the vacuum distillation column 56 via the pipe 57. The pipe 57 is provided with the heating unit 55. The heating unit 55 heats the atmospheric residual oil. The temperature of the mixture after heating may range from 300 degrees Celsius to 400 degrees Celsius, for example. The vacuum distillation column 56 distills the atmospheric residual oil, which is heated by the heating unit 55, under a reduced pressure. The vacuum distillation column 56 includes a plurality of trays, and is capable of separating hydrocarbons according to the carbon number by extracting a liquid component from each tray. The vacuum unit, which is omitted in the illustration, discharges the gas inside the vacuum distillation column 56, and reduces a pressure inside the vacuum distillation column 56. For example, the vacuum unit may include a vacuum pump.

For example, the vacuum distillation unit 54 distills the atmospheric residual oil under a reduced pressure of, for example, approximately 0.01 atm to 0.2 atm. In the vacuum distillation unit 54, distillation allows for the separation of various fractions such as vacuum gas oil and vacuum residue. Vacuum gas oil may contain hydrocarbons having a boiling point ranging from 350 degrees Celsius to 550 degrees Celsius, for example. The vacuum residue refers to hydrocarbons that remain after distillation, and may contain hydrocarbons having a boiling point exceeding 550 degrees Celsius, for example.

In the present embodiment, with the reaction heat generated by generating hydrocarbons in the reaction unit 40, the distillation unit 50 distills hydrocarbons. Further, carbon dioxide absorbed in the carbon dioxide collection unit 20 is separated by a low-temperature heat medium that has a temperature lower than a high-temperature heat medium being a heat medium introduced to the distillation unit 50, as a result of consuming part of the reaction heat in the distillation unit 50

The reaction heat is transferred through the heat medium passing through a heat medium flow passage 60. The heat medium flow passage 60 connects the carbon dioxide collection unit 20, the reaction unit 40, and the distillation unit 50 to each other. Specifically, the heat medium flow passage 60 connects the reboiler 26 of the carbon dioxide collection unit 20, the reaction unit 40, the heating unit 52 of the distillation unit 50, and the heating unit 55 of the distillation unit 50 to each other.

In the reaction unit 40, the reaction heat generated by generating hydrocarbons and the heat of the heat medium are exchanged. With this, the heat medium is heated by extracting the reaction heat, and the raw material or the product in the reaction unit 40 is cooled. The reaction where hydrocarbons are generated from the raw material containing hydrogen and carbon dioxide is an exothermic reaction. Thus, the heat medium extracts the reaction heat generated by generating hydrocarbons, and thereby the reaction of hydrocarbons can proceed advantageously.

Heating of the heat medium with the reaction heat may be performed inside the reaction unit 40. For example, when the reaction unit 40 includes a shell-and-tube type reactor, the heat medium may be heated by causing the heat medium to pass through the outside of the reaction tube in the shell. Further, the reaction product having the reaction heat passes through the cooling unit 42, and hence the heat medium may be heated by causing the heat medium to pass through the cooling unit 42. The high-temperature heat medium heated by the reaction heat is introduced to the distillation unit 50.

In the distillation unit 50, the heat of the high-temperature heat medium and the heat of hydrocarbons introduced to the distillation unit 50 are exchanged. Further, the distillation unit 50 distills hydrocarbons by the high-temperature heat medium. With this, the reaction heat can be used for distillation, and an energy required for distillation can be reduced. The heat medium is only required to be supplied to at least one of the atmospheric distillation unit 51 and the vacuum distillation unit 54. In other words, the heat medium may be supplied to any one of the atmospheric distillation unit 51 and the vacuum distillation unit 54, and may be supplied to both the atmospheric distillation unit 51 and the vacuum distillation unit 54. In the present embodiment, in the heat medium flow passage 60, the heating unit 52 and the heating unit 55 are provided in parallel to each other. The heat medium transferred from the reaction unit 40 is split and supplied to the heating unit 52 and the heating unit 55. The low-temperature heat medium whose reaction heat is partially consumed in the heating unit 52 and the low-temperature heat medium whose reaction heat is partially consumed in the heating unit 55 join to be introduced to the reboiler 26 of the carbon dioxide collection unit 20.

In the carbon dioxide collection unit 20, the heat of the low-temperature heat medium and the heat of the absorption solution can be exchanged. With this, in the reboiler 26, the absorption solution is heated. Thus, separation of carbon dioxide absorbed into the absorption solution can be promoted. Further, the low-temperature heat medium is cooled and introduced to the reaction unit 40.

As the heat medium, a publicly known heat medium such as steam and oil may be used. When oil is used as the heat medium, handling thereof is relatively easy, and the device configuration can be simplified. Further, when steam is used as the heat medium, steam is resistant to degradation by heat and oxidation, and cost reduction can be achieved.

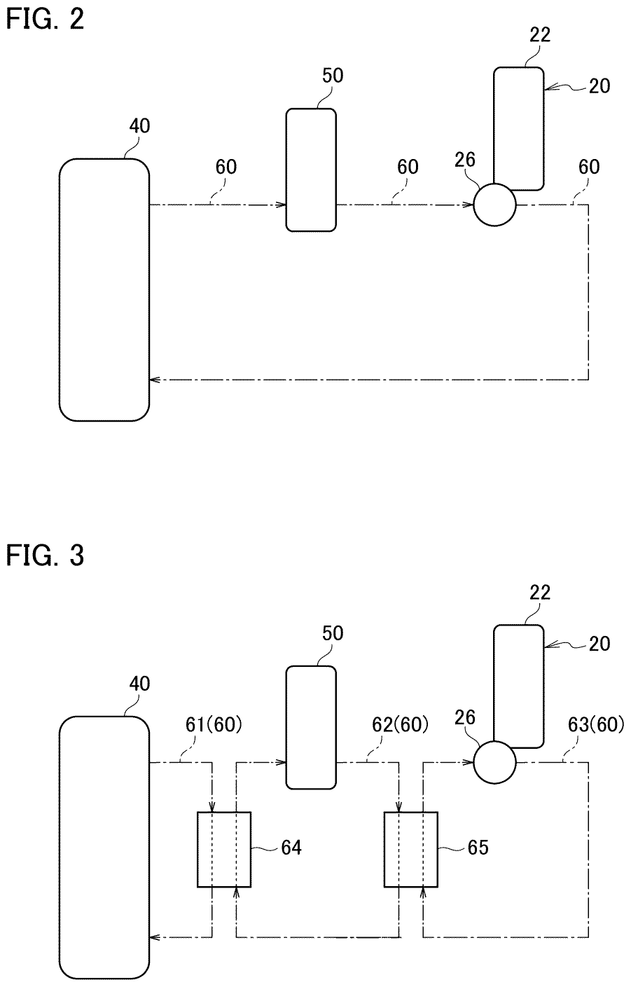

Note that, as illustrated in FIG. 2, in the present embodiment, description is made on an example in which the heat medium flow passage 60 connects the carbon dioxide collection unit 20, the reaction unit 40, and the distillation unit 50 to each other and the common heat medium circulates through the carbon dioxide collection unit 20, the reaction unit 40, and the distillation unit 50. However, the heat medium flow passage 60 is not limited to this example, and different heat media may pass through the heat medium flow passage 60.

For example, as illustrated in FIG. 3, the heat medium flow passage 60 may include a first circulation flow passage 61, a second circulation flow passage 62, and a third circulation flow passage 63. Further, the reaction system 1 may include a first heat exchanger 64 and a second heat exchanger 65. The first circulation flow passage 61 connects the reaction unit 40 and the first heat exchanger 64 to each other. A first heat medium flows through the first circulation flow passage 61, and the first heat medium circulates through the reaction unit 40 and the first heat exchanger 64. The second circulation flow passage 62 connects the distillation unit 50, the first heat exchanger 64, and the second heat exchanger 65 to each other. A second heat medium flows through the second circulation flow passage 62, and the second heat medium circulates through the distillation unit 50, the second heat exchanger 65, and the first heat exchanger 64 in the stated order. The third circulation flow passage 63 connects the reboiler 26 of the carbon dioxide collection unit 20 and the second heat exchanger 65 to each other. A third heat medium flows through the third circulation flow passage 63, and the third heat medium circulates through the reboiler 26 of the carbon dioxide collection unit 20 and the second heat exchanger 65.

The first heat medium is heated by the reaction heat generated by generating hydrocarbons in the reaction unit 40. The first heat medium and the second heat medium are subjected to heat exchange by the first heat exchanger 64, and the second heat medium is heated. The second heat medium heated by the first heat exchanger 64 is supplied as the high-temperature heat medium to the distillation unit 50, and hydrocarbons are distilled in the distillation unit 50. The second heat medium is transferred from the distillation unit 50 to the second heat exchanger 65, and the heat medium whose reaction heat is partially consumed in the distillation unit 50 and the third heat medium are subjected to heat exchange. The third heat medium (the low-temperature heat medium) heated by the second heat exchanger 65 is introduced to the reboiler 26 of the carbon dioxide collection unit 20, and carbon dioxide absorbed in the carbon dioxide collection unit 20 is separated. Meanwhile, the second heat medium cooled by the second heat exchanger 65 is supplied to the first heat exchanger 64, and is heated by the first heat medium in the first heat exchanger 64 as described above.

The first heat medium, the second heat medium, and the third heat medium may be the same heat medium, or may be different heat media. Further, in place of the first circulation flow passage 61 and the second circulation flow passage 62, a fourth circulation flow passage that connects the reaction unit 40 and the distillation unit 50 to each other may be used. In this case, the heat medium in the third circulation flow passage 63 and the heat medium in the fourth circulation flow passage are subjected to heat exchange by the second heat exchanger 65. Similarly, in place of the second circulation flow passage 62 and the third circulation flow passage 63, a fifth circulation flow passage that connects the distillation unit 50 and the carbon dioxide collection unit 20 to each other may be used. In this case, the heat medium in the first circulation flow passage 61 and the heat medium in the fifth circulation flow passage are subjected to heat exchange by the first heat exchanger 64.

With the configuration described above, by the reaction heat generated by generating hydrocarbons in the reaction unit 40, hydrocarbons are distilled in the distillation unit 50, and part of the reaction heat is consumed in the distillation unit 50. Further, carbon dioxide absorbed or adsorbed in the carbon dioxide collection unit 20 is separated by the low-temperature heat medium that has a temperature lower than the high-temperature heat medium being a heat medium introduced to the distillation unit 50, as a result of consuming part of the reaction heat in the distillation unit 50.

Note that, in the present embodiment, description is made on an example in which the carbon dioxide collection unit 20 collects carbon dioxide by a chemical absorption process. Specifically, description is made on an example in which the carbon dioxide collection unit 20 includes the absorption unit 21 that absorbs carbon dioxide into the absorption solution and the separation unit 22 that separates carbon dioxide absorbed into the absorption solution from the absorption solution, the separation unit 22 separating carbon dioxide by the low-temperature heat medium. With this, a large amount of carbon dioxide can be collected. However, the carbon dioxide collection unit 20 may collect carbon dioxide by a solid absorption process or a physical adsorption process. In the solid absorption process, a solid absorbent agent is used to collect carbon dioxide. In the physical adsorption process, a solid adsorbent agent is used to collect carbon dioxide.

When carbon dioxide is collected by the solid absorption process, the carbon dioxide collection unit 20 may include an absorption unit and a separation unit. Carbon dioxide is supplied from the carbon dioxide generation source 10 to the absorption unit. Further, the absorption unit is in a low-temperature state, and then carbon dioxide and the solid absorbent agent are brought into contact with each other. With this, carbon dioxide can be absorbed into the solid absorbent agent. The solid absorbent agent into which carbon dioxide is absorbed is transferred to the separation unit, and carbon dioxide absorbed into the solid absorbent agent is separated by causing the separation unit to be in a high-temperature state. Note that the carbon dioxide collection unit 20 may include an absorption and separation unit that absorbs and separates carbon dioxide, instead of including the absorption unit and the separation unit that are independent from each other. When the absorption and separation unit is in a low-temperature state, and carbon dioxide and the solid absorbent agent are brought into contact with each other, carbon dioxide can be absorbed into the solid absorbent agent. Further, when the absorption and separation unit is in a high-temperature state, carbon dioxide absorbed into the solid absorbent agent can be separated.

The solid absorbent agent may include at least one of a porous material carrying a basic substance on its surface and a porous material whose surface is modified with a base. Such a material has a large surface area and high reactivity of the base with respect to carbon dioxide, and hence a larger amount of carbon dioxide can be absorbed. The porous material may contain at least one selected from a group consisting of zeolite, alumina, silica, resin, clay, and activated carbon. Further, the basic substance may contain at least one amine compound selected from a group consisting of primary amine compounds, secondary amine compounds, and tertiary amine compounds. Further, the base for modifying the surface of the porous material may be an amino group. Those materials can be obtained by immersing the porous material in the above-mentioned amine compound and drying the resultant, thereby supporting the basic substance on the surface of the porous material or modifying the surface of the porous material with the base. Alternatively, those materials can be obtained by modifying the porous material with the basic substance through a chemical reaction such as an alcohol elimination reaction between the porous material surface and the amine compound.

The solid absorbent agent may contain at least one selected from a group consisting of alkali metals and alkaline earth metals. Those materials are capable of absorbing carbon dioxide efficiently. The absorbent material containing alkali metals may contain at least one of carbonates of alkali metals and lithium-transition metal composite oxides. The absorbent material containing alkaline earth metals may contain oxides of alkaline earth metals.

When carbon dioxide is collected by the physical adsorption process, the carbon dioxide collection unit 20 may include an adsorption unit and a desorption unit. Carbon dioxide is supplied to the adsorption unit. Further, the adsorption unit is in a low-temperature state, and carbon dioxide and the solid adsorbent agent are brought into contact with each other. With this, carbon dioxide can be adsorbed to the solid adsorbent agent. The solid adsorbent agent to which carbon dioxide is adsorbed is transferred to the desorption unit, and carbon dioxide adsorbed to the solid adsorbent agent can be desorbed by causing the desorption unit to be in a high-temperature state. Note that the carbon dioxide collection unit 20 may include an adsorption and desorption unit that adsorbs and desorbs carbon dioxide, instead of including the adsorption unit and the desorption unit that are independent from each other. When the adsorption and desorption unit is in a low-temperature state, and carbon dioxide and the solid adsorbent agent are brought into contact with each other, carbon dioxide can be adsorbed to the solid adsorbent agent. Further, when the adsorption and desorption unit is in a high-temperature state, carbon dioxide adsorbed to the solid adsorbent agent can be desorbed.

The solid adsorbent agent may contain a porous material. The porous material may contain at least one selected from a group consisting of zeolite, alumina, silica, resin, clay, and activated carbon.

Further, in the present embodiment, description is made on an example in which the reaction unit 40 includes the fixed bed reactor. However, the reaction unit 40 may include a reactor other than the fixed bed reactor, in addition to the fixed bed reactor or in place of the fixed bed reactor. For example, the reaction unit 40 may include a reactor of at least one type selected from a group consisting of a fixed bed reactor, a slurry bed reactor, and a fluidized bed reactor.

The slurry bed reactor accommodates a slurry. The slurry contains liquid hydrocarbons and FT synthesis catalyst particles dispersed within the liquid hydrocarbons. Further, by introducing the raw material containing carbon dioxide and hydrogen to the slurry, hydrocarbons are produced. According to the slurry bed reactor, hydrocarbons with a larger carbon number can be generated. Thus, components such as diesel and kerosene can be generated efficiently through distillation.

Further, in the present embodiment, description is made on an example in which carbon dioxide that is collected in the carbon dioxide collection unit 20 is used as the raw material in the reaction unit 40. However, the reaction unit 40 may be independent from the carbon dioxide collection unit 20, and may use carbon dioxide that does not pass through the carbon dioxide collection unit 20, as the raw material.

Further, in the present embodiment, description is made on an example in which hydrocarbons that are generated in the reaction unit 40 are distilled in the distillation unit 50. However, the distillation unit 50 may be independent from the reaction unit 40, and may distill hydrocarbons that are not generated in the reaction unit 40. In other words, the mixture introduced to the distillation unit 50 may be crude oil, a reaction product acquired from an FT synthesis, or a mixture thereof.

Further, in the present embodiment, description is made on an example in which the distillation unit 50 includes the atmospheric distillation unit 51 and the vacuum distillation unit 54. However, the distillation unit 50 may include at least one of the atmospheric distillation unit 51 and the vacuum distillation unit 54. In this case, hydrocarbons that are introduced to at least one of the atmospheric distillation unit 51 and the vacuum distillation unit 54 may be distilled by the reaction heat. With this, in the atmospheric distillation unit 51 or the vacuum distillation unit 54, distillation of hydrocarbons can be promoted. Further, the distillation unit 50 may include a plurality of distillation columns, and hydrocarbons may be distilled in each of the distillation columns. For example, naphtha may be distilled in a first distillation column, and kerosene may be distilled in a second distillation column.

When heat required for distillation is not sufficient, the heat medium may be heated by combusting some of the hydrocarbons such as methane generated in the reaction unit 40. Hydrocarbons thus generated is carbon dioxide-free fuels, allowing operation without apparent increase in the atmospheric carbon dioxide level.

As described above, the reaction system 1 according to the present embodiment includes the carbon dioxide collection unit 20 that collects carbon dioxide by an absorption process or an adsorption process, the reaction unit 40 that generates hydrocarbons from the raw material containing hydrogen and carbon dioxide, and the distillation unit 50 that distills hydrocarbons. In the present embodiment, hydrocarbons are distilled in the distillation unit 50 by the reaction heat generated by generating hydrocarbons in the reaction unit 40. Further, carbon dioxide absorbed or adsorbed in the carbon dioxide collection unit 20 is separated by the low-temperature heat medium that has a temperature lower than the high-temperature heat medium being a heat medium introduced to the distillation unit 50, as a result of consuming part of the reaction heat in the distillation unit 50.

A releasing temperature required in the separation unit 22 is, for example, from 100 degrees Celsius to 120 degrees Celsius, and is lower than a temperature from 350 degrees Celsius to 450 degrees Celsius, which is required for distillation in the distillation unit 50. The reaction heat can be used at a temperature suitable for the carbon dioxide collection unit 20 and the distillation unit 50, and hence energy efficiency can be improved as compared to a case in which the reaction heat is individually used.

While some embodiments are described above, the embodiments may be changed or modified based on the contents disclosed above. All the constituent elements in the embodiments described above and all the features described in the claims may be individually extracted and combined with each other as long as they do not conflict with each other.

For example, the present disclosure can contribute to Goal 13 of the Sustainable Development Goals (SDGs), led by the United Nations, which aims to “take urgent action to combat climate change and its impacts”.

What is claimed is:

1. A reaction system, comprising:
a carbon dioxide collection unit configured to collect carbon dioxide by an absorption process or an adsorption process;
a reaction unit configured to generate hydrocarbons from a raw material containing hydrogen and carbon dioxide; and
a distillation unit configured to distill hydrocarbons, wherein
hydrocarbons are distilled in the distillation unit by reaction heat generated by generating hydrocarbons in the reaction unit, and carbon dioxide absorbed or adsorbed in the carbon dioxide collection unit is separated by a low-temperature heat medium that has a temperature lower than a high-temperature heat medium being a heat medium introduced to the distillation unit, as a result of consuming part of the reaction heat in the distillation unit.

2. The reaction system according to claim 1, wherein
the distillation unit distills hydrocarbons being generated in the reaction unit.

3. The reaction system according to claim 1, wherein
carbon dioxide contained in the raw material includes carbon dioxide being collected in the carbon dioxide collection unit.

4. The reaction system according to claim 1, wherein
the carbon dioxide collection unit includes:
an absorption unit configured to absorb carbon dioxide into an absorption solution; and
a separation unit configured to separate, from the absorption solution, carbon dioxide being absorbed into the absorption solution, and
the separation unit separates carbon dioxide by the low-temperature heat medium.

5. The reaction system according to claim 1, wherein
the reaction unit includes a fixed bed reactor.

6. The reaction system according to claim 1, wherein
the distillation unit includes at least one of an atmospheric distillation unit and a vacuum distillation unit, and distills hydrocarbons by the reaction heat, the hydrocarbons being introduced to at least one of the atmospheric distillation unit and the vacuum distillation unit.
